(19) United States
(12) Patent Application Publication
(10) Pub. No.: US 2018/0064319 A1
(43) Pub. Date: Mar. 8, 2018

(54) ELECTRONIC ENDOSCOPE SYSTEM

(71) Applicant: HOYA CORPORATION, Tokyo (JP)

(72) Inventor: Yoshihiro HAYASHI, Tokyo (JP)

(73) Assignee: HOYA CORPORATION, Tokyo (JP)

(21) Appl. No.: 15/316,721

(22) PCT Filed: Apr. 22, 2016
(86) PCT No.: PCT/JP2016/062743

§ 371 Date: Dec. 6, 2016

(30) Foreign Application Priority Data
May 21, 2015 (JP) 2015-103391

(51) Int. Cl. A61B 1/06; A61B 1/045

(52) U.S. Cl. CPC A61B 1/0638; A61B 1/05; A61B 1/0646; A61B 1/045

(57) ABSTRACT

An electronic endoscope system including a light source unit alternately repeats irradiation and non-irradiation of illumination light while alternately switching illumination light to be irradiated between first and second illumination lights which is different from at least one of a light amount and a spectral property from the first illumination light; an image pickup device that captures a subject illuminated with the illumination light; and an image pickup device control that reads, during a non-irradiation period of the illumination light following an irradiation period of the illumination light, charges which have been accumulated in the image pickup device during the irradiation period, and wherein the irradiation period of the first illumination light and the second illumination light are set to have different lengths depending on at least one of a difference in light amount and a difference in spectral property between the first illumination light and the second illumination light.

### TECHNICAL FIELD

The present invention relates an electronic endoscope system for observing a subject, such as, a lesion portion.

### BACKGROUND ART

In a medical device field, electronic endoscopes configured to facilitate diagnosis on a lesion portion by performing concurrent observations using illumination light with different wavelength bands and properties are known. For example, Japanese Patent Provisional Publication No. 2010-068992A (hereafter, referred to as “patent document 1”) describes a concrete configuration of an electronic endoscope system of this type.

The patent document 1 discloses an electronic endoscope system in which a subject is illuminated alternately with white normal light and special light with a wavelength band different from that of the normal light, and object light from the subject is detected by a CMOS type image sensor. In a CMOS type image sensor, a rolling shutter scheme is employed, and exposing of pixels and reading of image signals are sequentially performed for each line. Therefore, when the subject is illuminated by alternately switching the illumination light between the normal light and the special light, information obtained when the subject is illuminated with the normal light and information obtained when the subject is illuminated with the special light are mixed into pixel signals. In the electronic endoscope system described in the patent document 1, in order to prevent the information on the subject illuminated with different types of illumination light from being mixed into pixel signals, the illumination light is turned off at intervals of one frame and the pixel signals are read while the illumination light is turned off.

### SUMMARY OF THE INVENTION

There is a case where, when a subject is alternately illuminated with the normal light and the special light as illustrated in the patent document 1, the difference between the illuminance of the subject illuminated with the normal light and the illuminance of the subject illuminated with the special light becomes large. In the case where the difference in illuminance between two subjects is large, if the exposure is corrected to conform one of the subject images, a problem arises that the other of the subject images becomes overexposure or underexposure.

The present invention is made in view of the above described circumstances, and the object of the invention is to provide an electronic endoscope system configured such that when a subject is observed with illumination light having different properties, the electronic endoscope system is capable of capturing the subject in appropriate exposure for every property type of illumination light.

An electronic endoscope system according to an embodiment of the invention comprises: a light source unit configured to alternately repeat irradiation and non-irradiation of illumination light while alternately switching illumination light to be irradiated between first illumination light and second illumination light which is different in regard to at least one of a light amount and a spectral property from the first illumination light; an image pickup device that captures a subject illuminated with the illumination light; and an image pickup device control means that reads, during a non-irradiation period of the illumination light following an irradiation period of the illumination light, charges which have been accumulated in the image pickup device during the irradiation period of the illumination light. In this configuration, the irradiation period of the first illumination light and the irradiation period of the second illumination light are set to have different lengths depending on at least one of a difference in light amount and a difference in spectral property between the first illumination light and the second illumination light.

By thus differentiating the irradiation periods depending on the optical property of each of the first and second illumination light, it becomes possible to capture a subject image illuminated with the respective illumination light at appropriate exposure.

In an embodiment of the invention, for example, the light amount of the first illumination light may be larger than the light amount of the second illumination light. In this case, the irradiation period of the first illumination light may be shorter than the irradiation period of the second illumination light.

In an embodiment of the invention, for example, the irradiation period of the first illumination light may be shorter than the non-irradiation period of the illumination light.

In an embodiment of the invention, the irradiation period of the second illumination light may be longer than the non-irradiation period of the illumination light.

In an embodiment of the invention, the light source unit may comprise: a light source that emits white light; a rotating plate having at least a first filter which filters the white light to extract the first illumination light from the white light and a second filter which filters the white light to extract the second illumination light from the white light, the first filter and the second filter being arranged along a circumferential direction on the rotating plate such that the first filter and the second filter have angular ranges, along the circumferential direction, respectively corresponding to the irradiation period of the first illumination light and the irradiation period of the second illumination light; and a rotation driving unit configured to insert the first filter into an optical path of the white light during the irradiation period of the first illumination light and to insert the second filter into the optical path during the irradiation period of the second illumination light, by rotating the rotating plate.

In an embodiment of the invention, on the rotating plate, the first filter, a light shielding part which blocks the white light, the second filter and a light shielding part which blocks the white light may be arranged in the circumferential direction of the rotating filter such that the first filter, the light shielding part, the second filter and the light shielding part have angular ranges, in the circumferential direction, respectively corresponding to the irradiation period of the first illumination light, the non-irradiation period, the irradiation period of the second illumination light and the non-irradiation period. In this case, the rotation driving unit is configured to insert each of the light shielding parts into the optical path during the non-irradiation period by rotating the rotating plate.

The image pickup device may be, for example, a CMOS type image sensor.

According to embodiments of the invention, it becomes possible capture a subject in appropriate exposure for every property type of illumination light when the subject is observed with illumination light having different properties.

### BRIEF DESCRIPTION OF THE DRAWINGS

FIG. 1 is a block diagram illustrating a configuration of an electronic endoscope system according to an embodiment of the invention.

FIG. 2 is a front view of a rotating filter unit provided in a processor according to the embodiment of the invention.

FIG. 3 is a front view of a rotating filter unit provided in a processor of a conventional electronic endoscope system.

FIG. 4 is an explanatory illustration for explaining exposing timing and reading timing for a solid state image pickup device according to the conventional electronic endoscope system.

FIG. 5 is an explanatory illustration for explaining exposing timing and reading timing for a solid state image pickup device according to the embodiment of the invention.

FIG. 6 is an explanatory illustration for explaining exposing timing and reading timing for a solid state image pickup device according to the embodiment of the invention.

FIG. 7 is an explanatory illustration for explaining exposing timing and reading timing for a solid state image pickup device according to a variation 1 of the embodiment of the invention.

FIG. 8 is a front view of a rotating filter unit provided in a processor according to the variation 1 of the embodiment of the invention.

FIG. 9 is an explanatory illustration for explaining exposing timing and reading timing for a solid state image pickup device according to a variation 2 of the embodiment of the invention.

FIG. 10 is a front view of a rotating filter unit provided in a processor according to the variation 2 of the embodiment of the invention.

### EMBODIMENTS FOR CARRYING OUT THE INVENTION

In the following, an embodiment of the invention is described with reference to the accompanying drawings. In the following, an electronic endoscope system is explained as an embodiment of the invention by way of example.

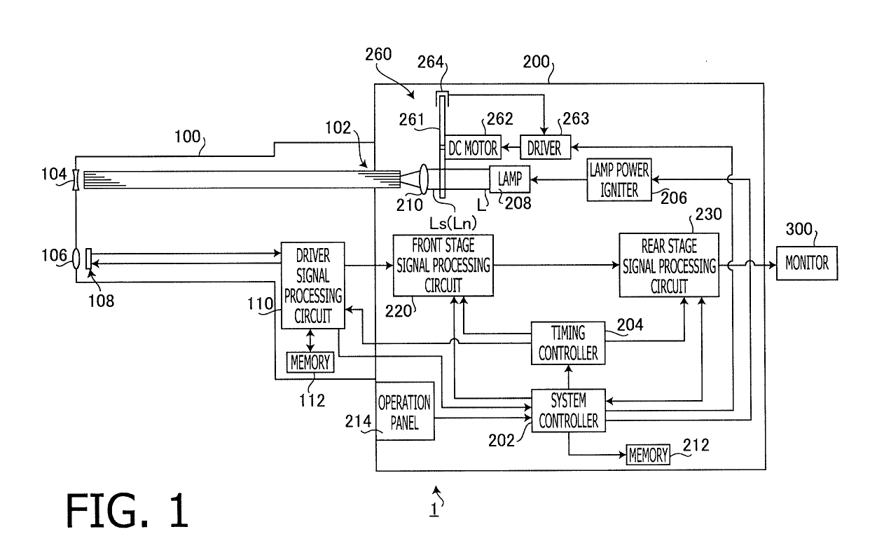

FIG. 1 is a block diagram illustrating a configuration of an electronic endoscope system 1 according to an embodiment of the invention. As shown in FIG. 1, the electronic endoscope system 1 includes an electronic scope 100, a processor 200 and a monitor 300.

The processor 200 includes a system controller 202 and a timing controller 204. The system controller 202 executes various programs stored in a memory 212, and totally controls the entire electronic endoscope system 1. Furthermore, the system controller 202 is connected to an operation panel 214. In accordance with instructions input by an operator through the operation panel 214, the system controller 202 alters operations and parameters for operations of the electronic endoscope system 1. The timing controller 204 outputs, to respective circuit in the electronic endoscope system 1, clock pulses for adjusting the operation timings of respective units.

After being activated by a lamp power igniter 206, a lamp 208 emits illumination light L. The lamp 208 is, for example, a high intensity lamp such as a xenon lamp, a halogen lamp, a mercury lamp, and a metal halide lamp, or a solid state light source such as an LED (Light Emitting Diode) and a laser diode. The illumination light L is light having a spectrum expanding principally from a visible light region to an infrared light region (or white light at least including the visible right region).

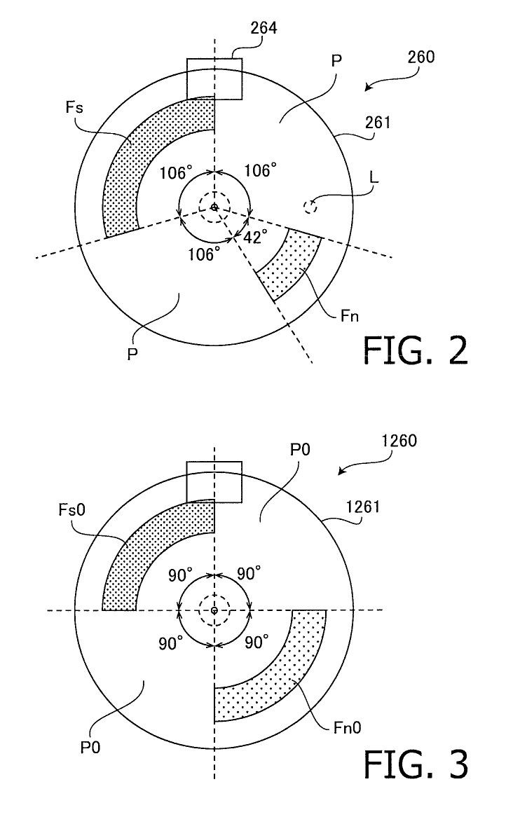

The illumination light L emitted from the lamp 208 is incident on a rotating filter unit 260. FIG. 2 is a front view of the rotating filter unit 260 viewed from a condenser lens 210 side. The rotating filter unit 260 includes a rotating turret 261, a DC motor 262, a driver 263, and a photo-interrupter 264. In FIG. 2, the illumination light L being incident on the rotating turret 261 is indicated by a dashed line. As shown in FIG. 2, in the rotating turret 261, a filter Fn for normal light (white light) and a filter Fs for special light are arranged sequentially in a circumferential direction. Each filter is formed in a shape of a fan, and is disposed to have an angular range corresponding to an exposure time and a reading time for charges regarding a solid state image pickup device 108 which is described later. Furthermore, a region P on the rotating turret 261 where no filter is provided serves as a light shielding plate which blocks the illumination light L.

The driver 263 drives the DC motor 262 under control of the system controller 202. By rotating the rotating turret 261 by the DC motor 262, the optical filters and the light shielding plates P are sequentially inserted into an optical path of the illumination light L. Thus, the illumination light L being incident on the rotating filter unit 260 from the lamp 208 is filtered by the respective optical filters, and one of the two types of illumination light (the normal light Ln and the special light Ls) having different spectrums is extracted at the timing synchronizing with the capturing. Between a period (an irradiation period of normal light) in which the normal light Ln is extracted and a period (an irradiation period of special light) in which the special light Ls is extracted, a light shielding period (non-irradiation period) in which the illumination light L is blocked by the light shielding plate P is provided. The phase of rotation and the rotational position of the rotating turret 261 are controlled by detecting an opening (not shown) formed in an outer peripheral portion of the rotating turret 261 by the photo-interrupter 264.

The normal light filter Fn is a dimmer filter which dims the illumination light L, and may be replaced with an opening (not having an optical filter) or a slit (not having an optical filter) also serving as an apertures top. The special light filter Fs has, for example, a spectral property suitable for capturing spectral images of a blood vessel structure near a surface layer (or a blood vessel structure in a deep layer, a particular lesion portion, etc.).

The illumination light L (the normal light Ln and the special light Ls) extracted by the rotating filter unit 260 is converged by the condenser lens 210 onto an entrance end face of an LCB (Light Carrying Bundle) 102, and enters the inside of the LCB 102.

The illumination light L (the normal light Ln and the special light Ls) which has entered the inside of the LCB 102 propagates through the inside of the LCB 102, and is emitted from an exit end face of the LCB 102 disposed in a tip portion of the electronic scope 100 to irradiate a subject through a light distribution lens 104. As a result, the subject is illuminated alternately with the normal light Ln and the special light Ls. The light returning from the subject being illuminated with the illumination light forms an optical image on a light-receiving surface of the solid state image pickup device 108 through an objective lens 106.

The solid state image pickup device 108 is a CMOS (Complementary Metal Oxide Semiconductor) type image sensor having a complementary color checkered pattern pixel arrangement. The solid state image pickup device 108 accumulates charges corresponding to a light amount of an optical image converged at each pixel on the light-receiving surface, generates and outputs pixel signals of yellow Ye, cyan Cy, green G and magenta Mg, mixes the pixel signals of two pixels arranged to adjoin with respect to each other in a vertical direction by adding together the pixel signals, and outputs the mixed signal. It should be noted that the solid state image pickup device 108 may be provided with a primary color filter (a Bayer arrangement filter).

The timing for switching between the normal light Ln and the special light Ls by the rotating filter unit 260 is in synchronizing with the exposing timing of the solid state image pickup device 108 and the reading timing for charges accumulated in the solid state image pickup device 108. As a result, the solid state image pickup device 108 outputs alternately the pixel signal of an observation image (a normal light observation image) of the subject illuminated with the normal light Ln and the pixel signal of an observation image (a special light observation image) of the subject illuminated with the special light Ls.

In a connection part of the electronic scope 100, a driver signal processing circuit 110 is provided. To the driver signal processing circuit 110, the pixel signals of the normal light observation image and the special light observation image are alternately input from the solid state image pickup device 108. The driver signal processing circuit 110 subjects the pixel signal input from the solid state image pickup device 108 to predetermined processing, and outputs the processed signal to a front stage signal processing circuit 220 of the processor 200.

Further, the driver signal processing circuit 110 accesses a memory 112 to read unique information of the electronic scope 100. The unique information stored in the memory 112 includes, for example, the pixel number and sensitivity of the solid state image pickup device 108, available frame rates, and a model number. The driver signal processing circuit 100 outputs the unique information read from the memory 112 to the system controller 202.

The system controller 202 executes various computations based on the unique information of the electronic scope 100 and generates control signals. Using the generated control signals, the system controller 202 controls operation and timings of various circuits in the processor 200 so that appropriate processing is performed for an electronic scope connected to the processor 200.

In accordance with the timing control performed by the system controller 202, the timing controller 204 supplies the driver signal processing circuit 110 with clock pulses. In accordance with the clock pulses supplied from the timing controller 204, the driver signal processing circuit 110 drives and controls the solid state image pickup device 108 at timings synchronizing with the frame rate of a video being processed on the processor 200 side.

The front stage signal processing circuit 220 subjects each of the pixel signals of the normal light observation image and the special light observation image to predetermined signal processing, such as color interpolation, a matrix operation and Y/C separation, and outputs the processed signal to a rear stage signal processing circuit 230.

The rear stage signal processing circuit 230 processes the pixel signal input from the front stage signal processing circuit 220 to generate image data for monitor representation, and converts the image data for monitor representation into a predetermined video format signal. The converted video format signal is outputted to the monitor 300. As a result, the special light observation image or the normal light observation image of the subject is displayed on a display screen of the monitor 300.

Hereafter, exposing timing and reading timing of charges (pixel signal) for a solid state image pickup device in a conventional endoscope system is explained.

FIG. 3 is a front view of a rotating filter unit 1260 provided in a processor of a conventional electronic endoscope system. The rotating filter unit 1260 includes a rotating turret 1261. In the rotating turret 1261, a normal light filter Fn0 and a special light filter Fs0 are arranged sequentially in a circumferential direction. Each optical filter is formed in a fan shape of which center angle is approximately 90 degrees, and the optical filters are disposed at rotationally-symmetrical positions with respect to a rotation axis. Furthermore, regions P0 in which no filter is provided in the rotating turret 1261 serve as light shielding plates for blocking illumination light. Therefore, by rotating the rotating turret 1261, states of irradiation of normal light, non-irradiation, irradiation of special light, and non-irradiation are switched at a predetermined frame rate ( 1/60 seconds in this embodiment).

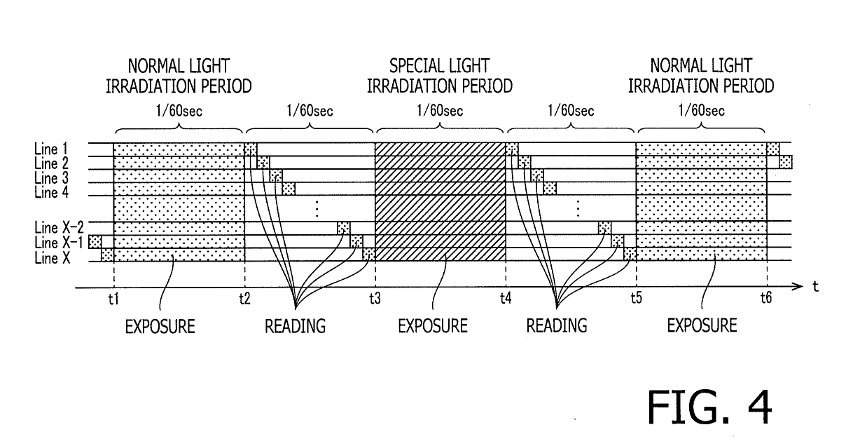

FIG. 4 is an explanatory illustration for explaining the exposing timing and the reading timing of charges (pixel signal) for a solid state image pickup device defined when a normal light observation image and a special light observation image are displayed to be arranged side by side on one screen in the conventional electronic endoscope system. The solid state image pickup device is a CMOS type image sensor, and employs a rolling shutter scheme for reading pixel signals.

On a light receiving surface of the solid state image pickup device, a plurality of pixels are arranged in a line and a plurality of such lines of pixels are arranged. The pixel signals are collectively read for each line. FIG. 4 illustrates the exposure timing and the reading timing of each line in a case where the solid state image pickup device has X lines of pixels including Line 1 to Line X.

The exposing timing of the solid state image pickup device and the reading timing of the pixel signal are in synchronization with rotation of the rotating turret 1261. Specifically, at time t1, irradiation of the normal light is started, and exposure of all the pixels of the image pickup device is started. The exposure of all the pixels is performed for 1/60 seconds until time t2. At time t2, the illumination light is blocked by the light shielding plate, and reading of charges accumulated during a time period from t1 to t2 in each pixel is performed sequentially for respective lines. Specifically, reading of the pixel signals is performed, in the order of ascending line number, while shifting the time for reading. The time required for reading of the pixel signals for all the pixels is 1/60 seconds. At time t3, irradiation of the special light is started, and exposure of all the pixels of the image pickup device is started. The exposure of all the pixels is performed for 1/60 seconds until time t4. At time t4, the illumination light is blocked, and reading of charges accumulated during a time period from t3 to t4 in each pixel is performed sequentially for respective lines.

By thus blocking irradiation of the illumination light during the time period in which the pixel signals of the subject illuminated with one of the normal light and the special light are read, the normal light observation image and the special light observation image are displayed on the monitor at 15 fps (frame per second) while preventing the information on the subject illuminated with the other of the normal light and the special light from being mixed into the pixel signals.

Since the normal light and the special light are different from each other in regard to the spectral property and the light amount, the illuminance of the subject illuminated with the normal light is different from the illuminance of the subject illuminated with the special light. However, in the conventional electronic endoscope system, the exposure time of the solid state image pickup device defined when the normal light is irradiated and the exposure time of the solid state image pickup device defined when the special light is irradiated are equal to each other. Furthermore, since the normal light and the special light are switched at a high speed cycle of 1/30 seconds, it is impossible to adjust an aperture stop or the gain for the pixel signal in an image processing circuit to conform the illuminance of the subject changing at a high speed. As a result, the amount of charges accumulated in the solid state image pickup device becomes different between the case where the normal light is irradiated and the case where the special light is irradiated. Therefore, when the aperture stop or the gain of the pixel signal is adjusted such that the subject image is obtained in an appropriate exposure for one of the normal light and the special light, the subject image for the other of the normal light and the special light may becomes overexposure or underexposure.

For this reason, in the electronic endoscope system 1 according to the embodiment is configured suitably for preventing occurrence of overexposure and underexposure of the subject image which is caused in the conventional electronic endoscope system.

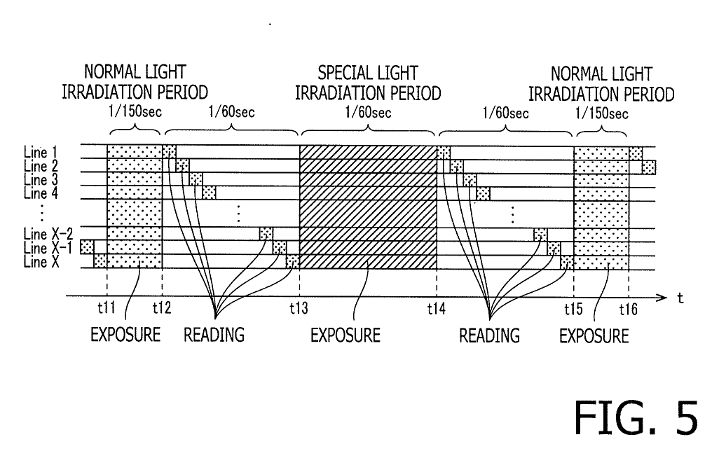

FIG. 5 is an explanatory illustration for explaining the exposing timing and the reading timing of charges (pixel signal) for the solid state image pickup device 108 defined when the normal light observation image and the special light observation image are displayed to he arranged side by side on one screen in the electronic endoscope system 1 according to the embodiment.

On the light receiving surface of the solid state image pickup device 108, a plurality of pixels are arranged in a line, and a plurality of such lines area arranged. The pixel signals are read collectively for each line. FIG. 5 illustrates the exposure time and the reading timing for each line in a case where the solid state image pickup device 108 includes X lines of Line 1 to Line X.

In this embodiment, the light amount of the normal light Ln is greater than the light amount of the special light Ln. Therefore, the exposure time for irradiation of the normal light Ln is set to be shorter than the exposure time for irradiation of the special light Ls. Specifically, the exposure time for irradiation of the special light Ln is 1/60 seconds which is equal to that defined in the conventional technology. On the other hand, the exposure time for irradiation of the normal light Ln is set to 1/150 seconds which is shorter by 1/100 seconds than the exposure time for irradiation of the special light Ls. Therefore, in this embodiment, a time period from a time (time t11) when the exposure for irradiation of the normal light Ln is started to a time (time t15) when next exposure for irradiation of the normal light Ln is started is approximately 57 ms ( 3/60 seconds + 1/150 seconds) which is shorter than that defined in the conventional technology ( 4/60≅67 ms).

In this embodiment, the rotation speed of the rotating turret 261 and the center angle of each filter are set to conform the exposure time and the reading timing of the solid state image pickup device 108. Specifically, the center angle of each of the special light filter Fs and the two light shielding plates is set to approximately 106 degrees, and the center angle of the normal light filter Fn is set to approximately 42 degrees. Furthermore, the rotation cycle of the rotating turret 261 is set to approximately 57 ms to conform the exposure cycle of the solid state image pickup device 108.

During the time period from time t11 to time t12, the normal light Ln is irradiated for 1/150 seconds, and all the pixels of the solid state image pickup device 108 are exposed. The timing when exposure is started and the timing when the exposure is finished are the same among the lines. During the time period from time t12 to time t13, the illumination light L is blocked, and charges accumulated in the respective pixels during the time period from time t11 to time t12 are read sequentially for each line. As shown in FIG. 5, the pixel signals are read sequentially in the order of ascending line number while shifting the time. In this embodiment, the time required to read the pixel signals from all the pixels is 1/60 seconds. During the time period from time t13 to time t14, the special light Ls is irradiated for 1/60 seconds, and all the pixels of the solid state image pickup device 108 are exposed. The timing when exposure is started and the timing when the exposure is finished are the same among the lines. During the time period from time t14 to time t15, the illumination light L is blocked, and charges accumulated in the respective pixels during the time period from time t13 to time t14 are read sequentially for each line. The pixel signals are read sequentially in the order of ascending line number while shifting the time. The time required to read the pixel signals from all the pixels is 1/60 seconds.

Thus, according to the embodiment, the exposure time for the solid state image pickup device 108 defined when the normal light Ln is irradiated is set to he shorter than the exposure time defined when the special light Ls is irradiated. Therefore, for both of the case where the normal light is irradiated and the case where the special light Ls is irradiated, the subject images captured with an appropriate exposure can be obtained.

Furthermore, in this embodiment, the exposure time for irradiation of the normal light Ln is set to be shorter than that defined in the conventional technology. Therefore, it becomes possible to increase the frame rate for the subject images obtained when the normal light Ln and the special light Ls are irradiated from approximately 67 ms (15 fps) to approximately 57 ms (approximately 18 fps).

A ratio between the irradiation period of the normal light Ln and the irradiation period of the special light Ls is set in accordance with a light amount ratio between the normal light Ln and the special light Ls. Therefore, the center angle of each filter and the irradiation period of each illumination light are not limited to the above described examples. For example, when the light amount of the special light Ls is greater than the light amount of the normal light Ln, the irradiation period of the special light Ls is set to be shorter than the irradiation period of the normal light Ln.

Furthermore, in this embodiment, the light shielding period (non-irradiation period) of the illumination light L is set to 1/60 seconds; however: embodiments are not limited to such an example. The light shielding period is set in accordance with a time required to read the pixel signals of the solid state image pickup device 108. The time required to read the pixel signals of the solid state image pickup device 108 differs depending on the specifications and operation modes of the solid state image pickup device 108. Therefore, the light shielding period of the illumination light L is set appropriately in accordance with the time required to read the solid state image pickup device 108.

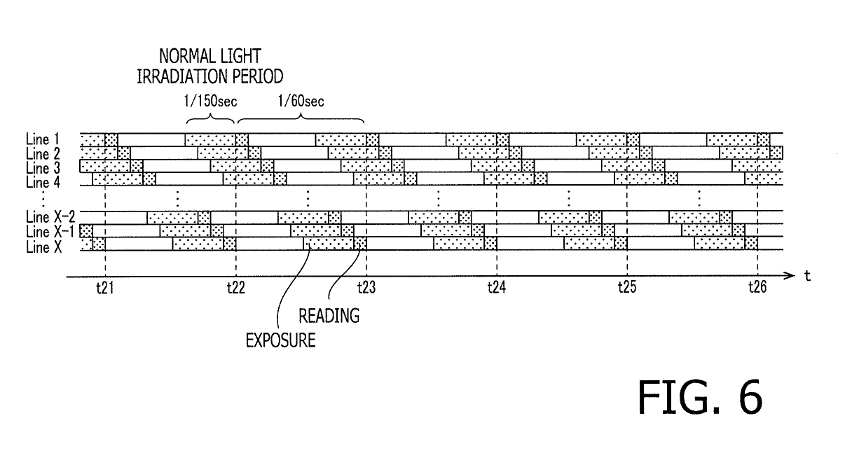

It should be noted that, in this embodiment, only a normal light observation image or only a special light observation image may be captured and displayed. FIG. 6 is an explanatory illustration for explaining the exposure timing and the reading timing for pixel signals when only a normal light observation image is displayed on the monitor 300.

When only a normal light observation image is to be captured, the information on the subject obtained when the subject is illumination with the normal light Ln and the information on the subject obtained when the subject is illumination with the special light Ls do not mix in contrast to the case where both of a normal light observation image and a special light observation image are captured simultaneously. Therefore, when only the normal light observation image is captured, there is no necessity to block the illumination light in accordance with the exposure time of each pixel and thereby the normal light is irradiated continuously. In a state where the normal light Ln is irradiated, the solid state image pickup device 108 exposes lines in the order of ascending line number while shifting the time. Regarding a pixel of each line, reading of a pixel signal is performed after exposure is performed for a predetermined time ( 1/150 seconds in the example in FIG. 6). The time required to read pixels for one frame is 1/60 seconds. Therefore, when only the normal light observation image or only the special light observation image is displayed, the frame rate for the subject image is approximately 17 ms (60 fps).

The foregoing is the explanation about the embodiment of the invention. The invention is not limited to the above described embodiment, but can be varied in various ways within the scope of the invention. For example, embodiments of the invention include a combination of embodiments explicitly described in this specification and embodiments easily realized from the above described embodiment.

### (Variation 1)

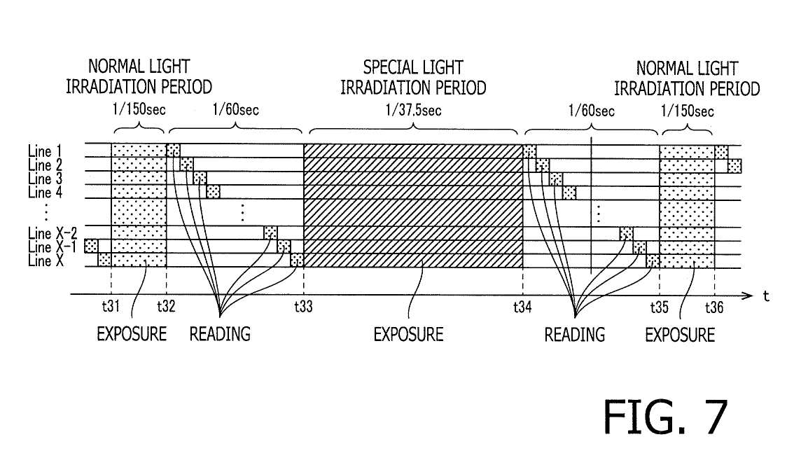

In the above described embodiment, the exposure time for irradiation of the normal light is set to 1/150 seconds which is shorter by 1/100 second than 1/60 seconds; however, the invention is not limited to such an example. For example, the exposure time for irradiation of the normal light Ln may be set to be shorter, and the exposure time for irradiation of the special light Ls may be set to be longer. FIG. 7 is an explanatory illustration for explaining the exposing timing and the reading timing for pixel signals of the solid state image pickup device 108 when the special light observation image and the normal light observation image are displayed side by side on one screen in the variation 1.

In the variation 1, the light shielding period is 1/60 seconds as in the case of the above described embodiment. The exposure time defined when the normal light Ln is irradiated is 1/150 seconds which is shorter by 1/100 seconds than 1/60 seconds. The exposure time for irradiation of the special light Ls is 1/37.5 seconds which is longer by 1/100 seconds than 1/60 seconds. Therefore, in this variation 1, the time elapsed from a time when exposure by irradiation of the normal light Ln is started (a time t31) to a time when next exposure by irradiation of the normal light Ln is started (a time t35) is approximately 67 ms, and the refresh rate of each observation image is 15 fps which is equal to that of the conventional technology.

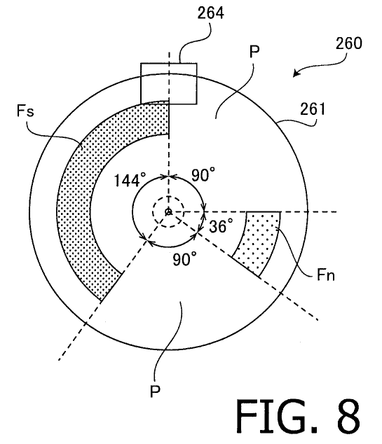

FIG. 8 is a front view of the rotating turret 261 according to the variation 1. A center angle of each filter provided in the rotating turret 261 according to the variation 1 is set to conform the reading timing of the solid state image pickup device 108. Specifically, the center angle of each of the two light shielding regions is 90 degrees, a center angle of for the normal light filter is 36 degrees, and a center angle of the special light filter is degrees.

Thus, in this variation 1, the exposure time for irradiation of the normal light Ln is set to be shorter, and the exposure time for irradiation of the special light Ls is set to be longer. Accordingly, even when the light amount difference (the light amount ratio) between the normal light Ln and the special light Ls is large, subject images can be obtained with appropriate exposure for both of the case where the normal light Ln is irradiated and the case where the special light Ls is irradiated.

### (Variation 2)

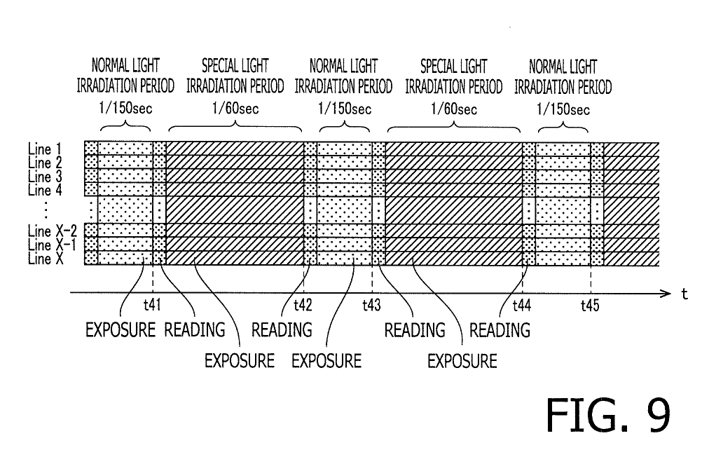

In the above described embodiment, a CMOS type image sensor is used as the solid state image pickup device 108; however, the present invention is not limited to such an example. For example, a CCD (Charge Coupled Device) type image sensor may be used as the solid state image pickup device 108. In a CCD type image sensor, a global shutter scheme in which pixel signals for all the pixels are read simultaneously is employed. FIG. 9 is an explanatory illustration for explaining the exposing timing and the reading timing for pixel signals of the solid state image pickup device 108 when the special light observation image and the normal light observation image are displayed side by side on one screen in the case where a CCD type image sensor is used as the solid state image pickup device 108.

As shown in FIG. 9, in the variation 2, the exposure time for irradiation of the normal light Ln is 1/150 seconds, and the exposure time for irradiation of the special light Ls is 1/60 seconds. When a CCD type image sensor is used as the solid state image pickup device 108, pixel signals are read simultaneously from all the pixels. Therefore, a time required to read the pixel signals from all the pixels is shorter than that required when a CMOS type image sensor is used. Accordingly, the light shielding time for the illumination light L is sufficiently smaller than the irradiation time of the normal light Ln and the irradiation time of the special light Ls.

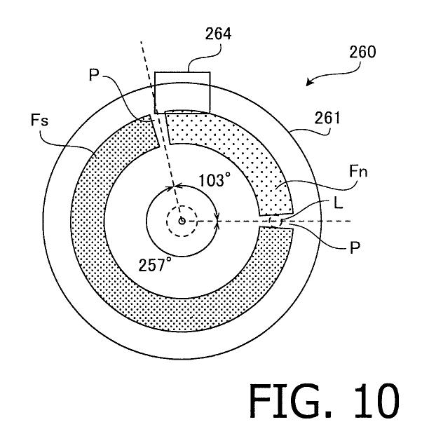

FIG. 10 is a front view of the rotating turret 261 according to the variation 2. A center angle of each filter provided in the rotating turret 261 according to the variation 2 is set to conform the reading timing of the solid state image pickup device 108. Specifically, the center angle of a normal light optical filter is approximately 103 degrees, the center angle of a special light optical filter is approximately 257 degrees. Furthermore, a light shielding plate P is provided within a small angular range in a boundary region between the normal light optical filter Fn and the special light optical filter Fs. The size of each light shielding plate P is set to be larger than the size of the illumination light L being incident on the rotating turret 261 which is indicated by a dashed line. With this configuration, it becomes possible to prevent the normal light Ln and the special light Ls from being irradiated simultaneously.

When a CCD type image sensor is used as the solid state image pickup device 108, the frame rate for a subject image can be enhanced to the extent that a time required to read the pixel signals for all the pixels is decreased in this variation.

1. An electronic endoscope system, comprising:
a light source unit configured to alternately repeat irradiation and non-irradiation of illumination light while alternately switching illumination light to be irradiated between first illumination light and second illumination light which is different in regard to at least one of a light amount and a spectral property from the first illumination light;
an image pickup device that captures a subject illuminated with the illumination light; and
an image pickup device control unit that reads, during a non-irradiation period of the illumination light following an irradiation period of the illumination light, charges which have been accumulated in the image pickup device during the irradiation period of the illumination light,
wherein the irradiation period of the first illumination light and the irradiation period of the second illumination light are set to have different lengths depending on at least one of a difference in light amount and a difference in spectral property between the first illumination light and the second illumination light.

2. The electronic endoscope system according to claim 1,
wherein:
the light amount of the first illumination light is larger than the light amount of the second illumination light; and
the irradiation period of the first illumination light is shorter than the irradiation period of the second illumination light.

3. The electronic endoscope system according to claim 1,
wherein the irradiation period of the first illumination light is shorter than the non-irradiation period of the illumination light.

4. The electronic endoscope system according to claim 1,
wherein the irradiation period of the second illumination light is longer than the non-irradiation period of the illumination light.

5. The electronic endoscope system according to claim 1,
wherein the light source unit comprises:
a light source that emits white light;
a rotating plate having at least a first filter which filters the white light to extract the first illumination light from the white light and a second filter which filters the white light to extract the second illumination light from the white light, the first filter and the second filter being arranged along a circumferential direction on the rotating plate such that the first filter and the second filter have angular ranges, along the circumferential direction, respectively corresponding to the irradiation period of the first illumination light and the irradiation period of the second illumination light; and
a rotation driving unit configured to insert the first filter into an optical path of the white light during the irradiation period of the first illumination light and to insert the second filter into the optical path during the irradiation period of the second illumination light, by rotating the rotating plate.

6. The electronic endoscope system according to claim 5,
wherein:
on the rotating plate, the first filter, a first light shielding part which blocks the white light, the second filter and a second light shielding part which blocks the white light are arranged in the circumferential direction of the rotating filter such that the first filter, the first light shielding part, the second filter and the second light shielding part have angular ranges, in the circumferential direction, respectively corresponding to the irradiation period of the first illumination light, the non-irradiation period, the irradiation period of the second illumination light and the non-irradiation period; and
the rotation driving unit is configured to insert each of the first light shielding parts and the second light shielding part into the optical path during the non-irradiation period by rotating the rotating plate.

7. The electronic endoscope system according to claim 1, wherein the image pickup device is a CMOS type image sensor.

8. The electronic endoscope system according to claim 1, wherein exposure time of the image pickup device for irradiation of the first illumination light is shorter than exposure time of the image pickup device for irradiation of the second illumination light.
